(19) United States
(12) Patent Application Publication
(10) Pub. No.: US 2016/0304932 A1
(43) Pub. Date: Oct. 20, 2016

(54) METHOD AND A KIT FOR THE DIAGNOSIS OF HEPATIC DISEASE UTILIZING THE SULFYDRYL-OXIDIZING PROPERTY OF SERUM

(71) Applicant: Anhui Agricultural University, Anhui (CN)

(72) Inventors: Jinsong Zhang, Anhui (CN); Kang Sun, Anhui (CN)

(21) Appl. No.: 15/087,842

(22) Filed: Mar. 31, 2016

(30) Foreign Application Priority Data
Apr. 16, 2015 (CN) 201510181394.X

(51) Int. Cl. C12Q 1/26; C12Q 1/28

(52) U.S. Cl. CPC C12Q 1/26; C12Q 1/28; G01N 2800/085; G01N 2800/26; G01N 2333/90212; G01N 2800/7028

(57) ABSTRACT

The present invention refers to a method and a kit for the diagnosis of a hepatic disease utilizing the sulfydryl-oxidizing property of serum. Sulfydryl can be oxidized by serum, due to the QSOXs and other macromolecules or small molecules in serum. The specific activity of QSOXs in serum can be detected by fluorescent quantification using Amplex UltraRed (AUR), horseradish perioxidase, dithiothreitol, and tween as key reagents. The total dithiol oxidated capacity of the serum can be detected by colorimetric quantification using dithiothreitol, dithio-bis-nitrobenzoic acid, and guanidine hydrochloride as key reagents. The present invention shows that the specific activity of QSOXs in serum and the total dithiol oxidated capacity of the serum can be used for the diagnosis of hepatic disease, wherein the total dithiol oxidated capacity of the serum shows better diagnostic effect. The present invention also refers to a kit for the diagnosis of a hepatic disease, comprising a key reagent including phosphate buffer, Amplex UltraRed (AUR), horseradish perioxidase, dithiothreitol, and tween; or including guanidine hydrochloride, dithio-bis-nitrobenzoic acid, and dithiothreitol, etc.

### CROSS-REFERENCE TO RELATED APPLICATION

This application claims priority to China application no. 201510181394.X filed on Apr. 16, 2015, which is hereby incorporated by reference in its entirety.

### FIELD OF THE INVENTION

The present invention refers to a method for the diagnosis of a hepatic disease utilizing the sulfydryl-oxidizing property of serum. In particular, the present invention refers to the diagnosis of a hepatic disease using the specific activity of Quiescin sulfhydryl oxidases (QSOXs) in serum, or the total dithiol oxidated capacity (TDOC) of the serum as a biological index. The present invention also refers to a kit for the diagnosis of a hepatic disease.

### BACKGROUND

Common hepatic diseases include hepatitis, liver cirrhosis (LC), primary hepatocellular carcinoma (HCC), and the like. There are a great number of serum indexes for the diagnosis of hepatic disease. Glutamic-pyruvic transaminase (GPT), glutamic-oxalacetic transaminase (GOT), alpha fetoprotein(AFP), and the ratio of albumin (ALB) to globulin (GLOB) are the common diagnostic indexes. However, the sensitivity and specificity of these indexes include limitations, and new serum index is needed to improve the diagnostic effect.

Quiescin sulfhydryl oxidases (QSOXs) can catalyze the reaction of sulfydryl of cysteine residue and oxygen to form disulfide linkage and hydrogen peroxide. Currently, the specific activity of QSOXs in serum can be detected by fluorescent quantification using Amplex UltraRed (AUR), horseradish perioxidase, dithiothreitol, and tween as key reagents. QSOXs activity has been detected in human and murine sera with this method, of which the use in diagnosis of a disease has not been reported. Serum can oxidize sulfydryl due to not only QSOXs but also other macro and small molecules, which may facilitate the oxidization of sulfydryl. The total dithiol oxidated capacity of the serum can be detected by colorimetry using dithiothreitol, dithio-bis-nitrobenzoic acid(DTNB), and guanidine hydrochloride as key reagents, but the use of this method in diagnosis of a disease has not been reported yet.

### SUMMARY

The present invention refers to a method for the diagnosis of a hepatic disease, characterized in utilizing the sulfydryl-oxidizing property of serum. The accuracy of the diagnosis of a hepatic disease can be effectively increased by using the method of the present invention, and hepatic diseases and non-hepatic diseases can be effectively distinguished.

The hepatic diseases include, but are not limited to, hepatocellular carcinoma, hepatitis B virus (HBV), and liver cirrhosis (LC). The non-hepatic diseases include, but are not limited to, hypertension, diabetes mellitus, atherosclerosis, gastric cancer, colorectal cancer, lung cancer, breast cancer, ovarian cancer and prostatic cancer.

The sulfydryl-oxidizing property of serum can be measured by the detection of the specific activity of QSOXs in serum, which is achieved by fluorescent quantification, or by the detection of the total dithiol oxidated capacity of the serum, which is achieved by colorimetric quantification.

The fluorescent quantification comprises the use of the following key reagents^{.} Amplex UltraRed (AUR), horseradish perioxidase, dithiothreitol, and tween.

The colorimetric quantification comprises the use of the following key reagents: dithiothreitol, dithio-bis-nitrobenzoic acid, and guanidine hydrochloride.

In the present invention, both the specific activity of QSOXs in serum and the total dithiol oxidated capacity of the serum can be used as a biological index for the diagnosis of a hepatic disease. The total dithiol oxidated capacity shows better results, the detection of the total dithiol oxidated capacity of the serum can further increase the accuracy of the diagnosis of a hepatic disease. In addition, a non-hepatic disease does not substantially influence the total dithiol oxidated capacity, and thus can be distinguished from a hepatic disease.

The present invention also refers to a kit for the diagnosis of a hepatic disease, characterized in comprising a reagent for the detection of the degree of the sulfydryl oxidization by serum.

Preferably, the reagent is selected from the reagent for the detection of the specific activity of QSOXs in serum and the reagent for the detection of the total dithiol oxidated capacity of the serum.

Preferably, the hepatic disease includes, but is not limited to, hepatocellular carcinoma, hepatitis B virus, and liver cirrhosis.

The Kit is characterized in comprising Amplex UltraRed (AUR), horseradish perioxidase, dithiothreitol, and tween used in the fluorescent quantification for the detection of the specific activity of QSOXs in serum; or comprising guanidine hydrochloride, dithio-bis-nitrobenzoic acid, and dithiothreitol used in the colorimetric quantification for the detection of the total dithiol oxidated capacity of the serum. Preferably, the reagent includes 50 mM phosphate buffer (pH7.5, containing 1 mM EDTA), 25 μM Amplex UltraRed (AUR), 0.125 μM horseradish perioxidase, 50 μM dithiothreitol, and 1.25% (weight) tween; or includes 6.6 M guanidine hydrochloride solution, 10 mM dithio-bis-nitrobenzoic acid, and 1 mM dithiothreitol.

More preferably, the reaction system for the detection of the specific activity of QSOXs in serum comprises 50 mM phosphate buffer (pH7.5, containing 1 mM EDTA), 10 μM Amplex UltraRed (AUR), 0.05 μM horseradish perioxidase, 50 μM dithiothreitol, and 0.5% (weight) tween; or the reaction system for the detection of the total dithiol oxidated capacity of the serum comprises reaction liquid of 200 mM HEPES buffer (pH7.5, containing 1 Mm dithiothreitol), stopping solution of 6.6 M guanidine hydrochloride solution (pH8.0, containing 200 mM Tris hydrochloride), and 10 mM dithio-bis-nitrobenzoic acid that is used as coloring solution.

The present invention also refers to the use of the kit for the detection of a serum index, including the specific activity of QSOXs in serum and the total dithiol oxidated capacity of the serum.

### BRIEF DESCRIPTION OF THE DRAWINGS

FIGS. 1A-1F show the serum indexes of the subjects having a hepatic disease. FIG. 1A: albumin/globulin; FIG. 1B: Glutamic-pyruvic transaminase; FIG. 1C: glutamic-oxalacetic transaminase; FIG. 1D: alpha fetoprotein; FIG. 1E: the specific activity of QSOXs in serum; and FIG. 1F: the total dithiol oxidated capacity of the serum. **: p<0.01, as compared to the control; ***: p<0.001, as compared to the control.

FIG. 2 shows the relevance between the specific activity of QSOXs in serum and the total dithiol oxidated capacity of the serum.

FIGS. 3A-3D show the hepatic disease-diagnostic effect of the serum indexes analyzed with ROC curve. FIG. 3A: hepatocellular carcinoma; FIG. 3B: hepatitis B virus; FIG. 3C: liver cirrhosis; and FIG. 3D: hepatic diseases (the incorporation of hepatocellular carcinoma, hepatitis B virus, and liver cirrhosis).

FIG. 4A shows a comparison of the total dithiol oxidated capacity of the serum in subjects having a hepatic disease with that in subjects having a non- hepatic disease; FIG. 4B shows the ROC curve analysis of the total dithiol oxidated capacity of the serum.

### DETAILED DESCRIPTION OF SPECIFIC EMBODIMENTS

The present invention is further described in the following examples in combination with the figures.

The examples of the present invention include the detection of the specific activity of QSOXs in serum by fluorescent quantification, the detection of the total dithiol oxidated capacity of the serum achieved by colorimetric quantification, and the detection of albumin, globulin, Glutamic-pyruvic transaminase, glutamic-oxalacetic transaminase, and alpha fetoprotein in serum with commercial kits, as well as the test of the hepatic disease-diagnostic effect of serum indexes with receiver operating characteristic curve (ROC curve) analysis.

The population, from which the sera tested in the examples are obtained, comprises 269 patients with a chronic hepatic disease (including 123 patients with hepatocellular carcinoma, 100 patients with hepatitis B virus, and 53 patients with liver cirrhosis), 548 patients with a cancer (including 101 patients with lung cancer, 91 patients with gastric cancer, 86 patients with Colorectal cancer, 105 patients with breast cancer, 66 patients with ovarian cancer and 99 patients with prostatic cancer), 91 patients with hypertension, 103 patients with diabetes mellitus, 100 patients with atherosclerosis, and 100 healthy controls. The detailed information of the population is shown in Table 1.

TABLE 1
The information of the population, from which
the tested sera are obtained.

| | | Male | | Female | |
|---|---|---|---|---|---|
| | | Popu- | | Popu- | |
| Patients | Age | lation | Percentage | lation | Percentage |
| Healthy control | 42.2 ± 14.6 | 54 | 54.0 | 46 | 46.0 |
| Hepatocellular | 61.6 ± 14.3 | 92 | 74.8 | 31 | 25.2 |
| carcinoma (HCC) | | | | | |
| Hepatitis B virus | 42.0 ± 13.1 | 67 | 72.0 | 26 | 28.0 |
| (HBV) | | | | | |
| Liver | 53.9 ± 12.7 | 39 | 73.6 | 14 | 26.4 |
| cirrhosis (LC) | | | | | |
| Hypertension | 68.5 ± 13.1 | 50 | 54.9 | 41 | 45.1 |
| Diabetes mellitus | 54.6 ± 13.1 | 69 | 67.0 | 34 | 33.0 |
| Atherosclerosis | 63.3 ± 14.0 | 48 | 48.0 | 52 | 52.0 |
| Lung cancer | 61.2 ± 12.3 | 62 | 61.4 | 39 | 38.6 |
| Gastric cancer | 63.3 ± 11.3 | 68 | 74.7 | 23 | 25.3 |
| Colorectal cancer | 63.9 ± 14.3 | 53 | 61.6 | 33 | 38.4 |
| Breast cancer | 52.5 ± 10.2 | 0 | 0.0 | 105 | 100.0 |
| Ovarian cancer | 47.0 ± 16.2 | 0 | 0.0 | 66 | 100.0 |
| Prostatic cancer | 75.2 ± 8.5 | 99 | 100.0 | 0 | 0.0 |

The detection of the specific activity of QSOXs in serum comprises the following steps: adding 80 μL operating solution (50 mM phosphate buffer (pH7.5), 25 μM Amplex UltraRed, 0.125 μM horseradish perioxidase, 1.25% tween and 2.5 mM EDTA) to a 96 -well plate at room temperature, adding 20 μL serum sample or normal saline (control), adding 100 μL dithiothreitol (100 μM) to start the reaction. The change in fluorescent intensity, with 544 nm excitation and 590 nm emission, within 10 minutes is detected. Standard curve is generated with the fluorescent intensity of hydrogen peroxide. 1 active unit means that 1 mol hydrogen peroxide can be generated in the reaction.

The detection of the total dithiol oxidated capacity of the serum comprises the following steps: mixing 100 μL serum with 50 μL dithiothreitol (1 mM) for 15 minutes at 37° C., adding 180 μL stopping solution (6.6 M guanidine hydrochloride), adding 20 μL coloring solution (10 mM DTNB), performing colorimetric analysis at 412 nm. The total dithiol oxidated capacity of the serum is calculated with absorption and the extinction coefficient of DTNB (13,600 M⁻¹ cm⁻¹). 1 active unit means that 1 nmol sulfydryl can be oxidized by the serum.

The common indexes for the diagnosis of a hepatic disease are detected with commercial kits. The concentration of albumin and globulin, and the activities of Glutamic-pyruvic transaminase and glutamic-oxalacetic transaminase are detected with an automatic biochemical analyzer (Roche Cobas C311) and commercial kits following the operating instructions. The level of alpha fetoprotein is detected with an immunoassay analyzer (Abbott, Chicago, Ill., USA) and a commercial kit following the operating instructions. If the concentration of alpha fetoprotein is shown greater than 80,000 ng/mL, it is considered as 80,000 ng/mL. All the detections are designed with parallel repeats.

All the data are shown as mean value±standard error. Mann-Whitney U test or 1-D variance Dunnett test is used for testing. The diagnostic effects are evaluated with the area under curve (AUC) of the receiver operating characteristic curve (ROC curve). The software SPSS 17.0 or GraphPad Prism 5.0 is used for analysis. The results are deemed as statistically different if the p-value is less than 0.05.

Results of the example are as follows.

1) Serum Indexes of the Patients with a Hepatic Disease

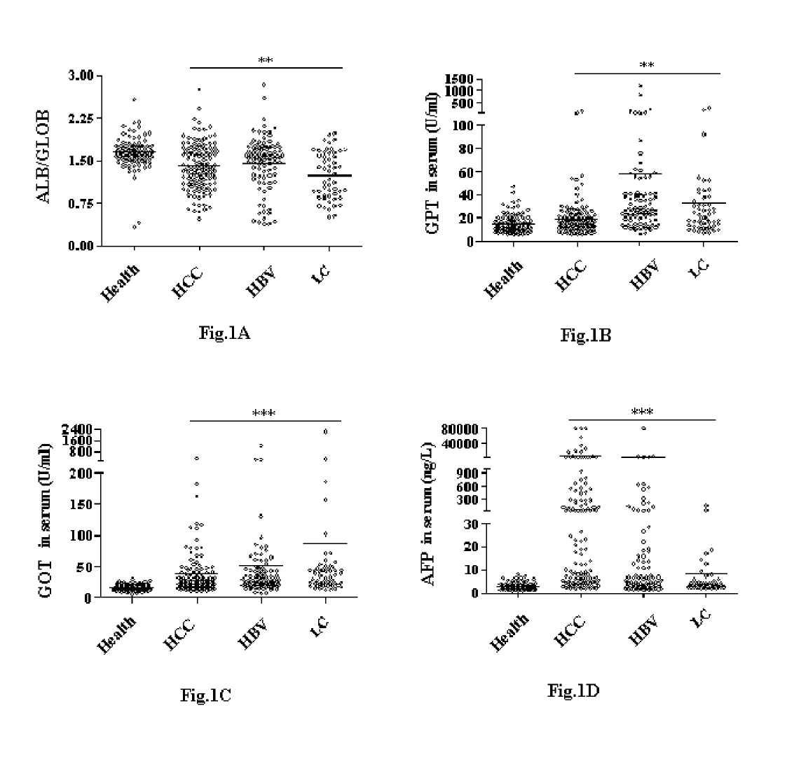

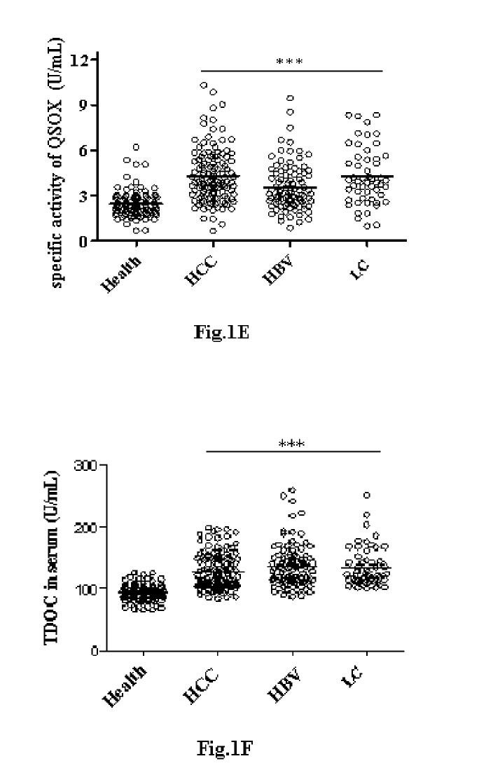

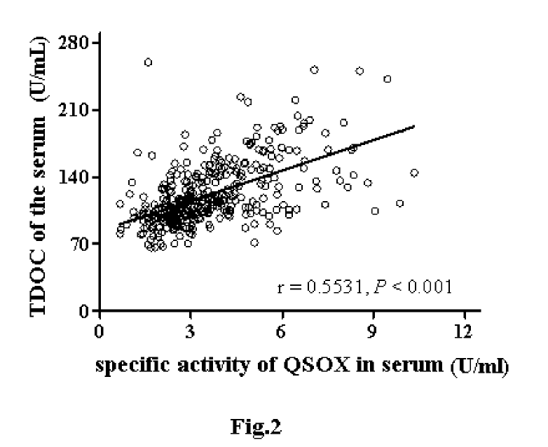

In the patients with hepatocellular carcinoma, hepatitis B virus, or liver cirrhosis, the ratio of albumin to globulin is significantly decreased, the activities of Glutamic-pyruvic transaminase and glutamic-oxalacetic transaminase are significantly increased, the level of alpha fetoprotein is significantly increased, and the specific activity of QSOXs in serum and the total dithiol oxidated capacity of the serum are significantly increased, as compared to the healthy controls (FIGS. 1A-1F). The specific activity of QSOXs in serum is significantly associated with the total dithiol oxidated capacity of the serum (r=0.5531) (FIG. 2), indicating that these two indexes are internally associated but not equal to each other.

2) Comparison Between Hepatic Disease-Diagnostic Effects of Various Indexes

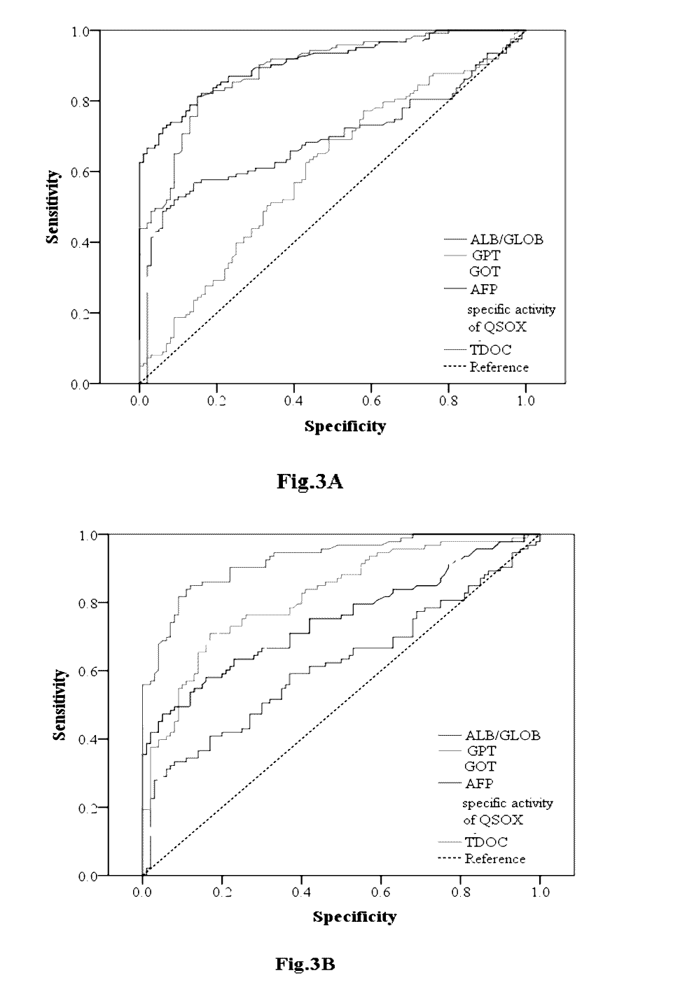

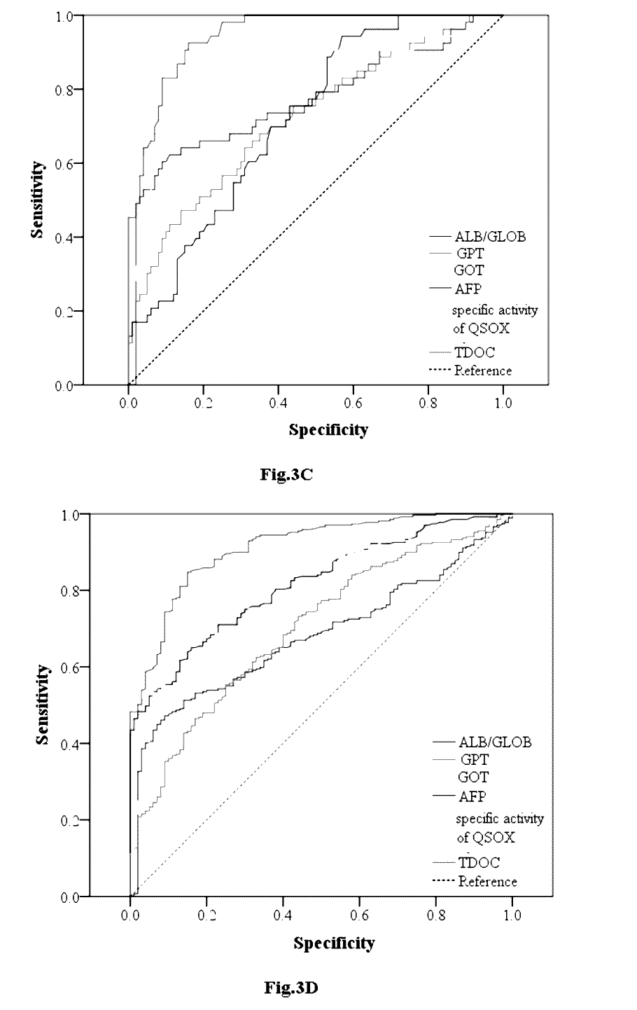

It is known that a greater AUC value means a better diagnostic effect. Table 2 shows that alpha fetoprotein is the best diagnostic index for hepatocellular carcinoma, and the total dithiol oxidated capacity of the serum is the best diagnostic index for other hepatic diseases or the combination of all the hepatic diseases. FIGS. 3A-3D shows the diagnostic effects analyzed with ROC curves, the results are identical to that shown in

Table 2.

TABLE 2
Comparison between hepatic disease-diagnostic effects of various indexes

| | | | | | | | The combination of | |
|---|---|---|---|---|---|---|---|---|
| | | | | | | | hepatocellular | |
| | | | | | | | carcinoma, hepatitis | |
| | Hepatocellular | | Hepatitis B | | Liver | | B virus and liver | |
| | carcinoma | | virus | | cirrhosis | | cirrhosis | |
| | | 95% | | 95% | | 95% | | 95% |
| | | Confidence | | Confidence | | Confidence | | Confidence |
| Variable | AUC | interval | AUC | interval | AUC | interval | AUC | interval |
| albumin/ | 0.690 | 0.620~0.761 | 0.616 | 0.535~0.697 | 0.769 | 0.682~0.856 | 0.680 | 0.626~0.735 |
| globulin | | | | | | | | |
| Glutamic- | 0.604 | 0.529~0.678 | 0.818 | 0.759~0.877 | 0.721 | 0.635~0.807 | 0.701 | 0.644~0.758 |
| pyruvic | | | | | | | | |
| transaminase | | | | | | | | |
| Glutamic- | 0.798 | 0.74~0.855 | 0.844 | 0.787~0.900 | 0.879 | 0.816~0.941 | 0.830 | 0.788~0.871 |
| oxalacetic | | | | | | | | |
| transaminase | | | | | | | | |
| Alpha | 0.908 | 0.871~0.945 | 0.749 | 0.679~0.819 | 0.720 | 0.640~0.800 | 0.816 | 0.773~0.859 |
| fetoprotein | | | | | | | | |
| The specific | 0.847 | 0.795~0.898 | 0.739 | 0.668~0.809 | 0.822 | 0.744~0.900 | 0.805 | 0.758~0.852 |
| activity of | | | | | | | | |
| QSOXs in | | | | | | | | |
| serum | | | | | | | | |
| The total | 0.888 | 0.847~0.930 | 0.926 | 0.890~0.961 | 0.946 | 0.915~0.978 | 0.913 | 0.882~0.943 |
| dithiol | | | | | | | | |
| oxidated | | | | | | | | |
| capacity of | | | | | | | | |
| the serum | | | | | | | | |

3) Comparison of the Total Dithiol Oxidated Capacity of the Serum (TDOC), In Patients with a Hepatic Disease to that in Patients with a Non-Hepatic Disease

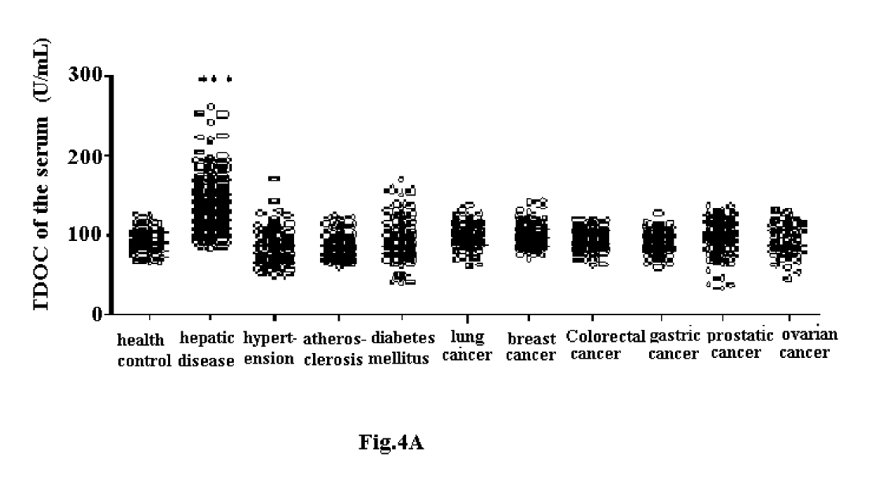

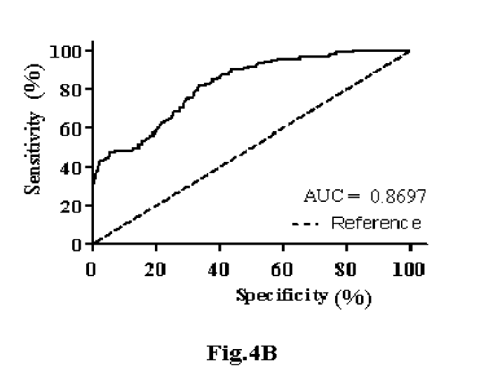

The above results show that the total dithiol oxidated capacity of the serum is an excellent index of for the diagnosis of a hepatic disease, but it is also important whether such the index will increase in the patients with other diseases. Therefore, we further compared the total dithiol oxidated capacity of the serum in the patients with hepatic and non-hepatic diseases. FIG. 4A shows that the total dithiol oxidated capacity of the serum in the patients with non-hepatic diseases is not significantly increased. Using the results in the patients with non-hepatic diseases as control, the patients with a hepatic disease can be clearly distinguished with ROC curve analysis, the AUC thereof reaches 0.8697 (FIG. 4B). These results indicate that the total dithiol oxidated capacity of the serum is suitable for the diagnosis of a hepatic disease. In conclusion, both the specific activity of QSOXs in serum and the total dithiol oxidated capacity of the serum can be used as a serum index for the diagnosis of a hepatic disease to increase the accuracy of the same, the latter shows a better effect. In addition, hepatic and non-hepatic diseases can be distinguished with the total dithiol oxidated capacity of the serum.

The example above does not to intend to limit the present invention, the diagnosis of other hepatic disease with the specific activity of QSOXs in serum or the total dithiol oxidated capacity of the serum shall also be within the scope of the present invention.

What is claimed is:

1. A method for the diagnosis of a hepatic disease utilizing the sulfydryl-oxidizing property of a serum.

2. The method of claim 1, wherein the hepatic disease includes, but is not limited to, hepatocellular carcinoma, hepatitis B virus, and liver cirrhosis.

3. The method of claim 1, comprising the detection of the specific activity of QSOXs in the serum, or the detection of the total dithiol oxidated capacity of the serum.

4. The method of claim 3, wherein the detection of the specific activity of QSOXs in the serum is achieved by fluorescent quantification comprising the use of the following key reagents: Amplex UltraRed (AUR), horseradish perioxidase, dithiothreitol, and tween; the detection of the total dithiol oxidated capacity of the serum is achieved by colorimetric quantification comprising the use of the following key reagents: dithiothreitol, dithio-bis-nitrobenzoic acid, and guanidine hydrochloride.

5. A kit for the diagnosis of a hepatic disease utilizing the sulfydryl-oxidizing property of a serum, characterized in comprising a reagent for the detection of the sulfydryl-oxidizing property of the serum.

6. The kit of claim 5, wherein the hepatic disease includes, but is not limited to, hepatocellular carcinoma, hepatitis B virus, and liver cirrhosis.

7. The kit of claim 5, wherein the reagent is selected from the reagent for the detection of the specific activity of QSOXs in the serum and the reagent for the detection of the total dithiol oxidated capacity of the serum.

8. The kit of claim 5, characterized in that the reagent includes phosphate buffer, Amplex UltraRed (AUR), horseradish perioxidase, dithiothreitol, and tween; or includes guanidine hydrochloride, dithio-bis-nitrobenzoic acid, and dithiothreitol.

9. The kit of claim 8, wherein the reagent includes 50 mM phosphate buffer (pH7.5, containing 1 mM EDTA), 25 μM Amplex UltraRed (AUR), 0.125 μM horseradish perioxidase, 125 μM dithiothreitol, and 1.25% (weight) tween; or includes 6.6 M guanidine hydrochloride solution, 10 mM dithio-bis-nitrobenzoic acid, and 1 mM dithiothreitol.

10. Use of a kit for the detection of a serum index, the serum index includes the specific activity of QSOXs in a serum and the total dithiol oxidated capacity of the serum; wherein the kit is for the diagnosis of a hepatic disease utilizing the sulfydryl-oxidizing property of the serum, the kit characterized in comprising a reagent for the detection of the sulfydryl-oxidizing property of the serum.

11. The use of a kit according to claim 10, wherein the hepatic disease includes, but is not limited to, hepatocellular carcinoma, hepatitis B virus, and liver cirrhosis.

12. The use of a kit according to claim 10, wherein the reagent is selected from the reagent for the detection of the specific activity of QSOXs in the serum and the reagent for the detection of the total dithiol oxidated capacity of the serum.

13. The use of a kit according to claim 10, characterized in that the reagent includes phosphate buffer, Amplex UltraRed (AUR), horseradish perioxidase, dithiothreitol, and tween; or includes guanidine hydrochloride, dithio-bis-nitrobenzoic acid, and dithiothreitol.

14. The use of a kit according to claim 13, wherein the reagent includes 50 mM phosphate buffer (pH7.5, containing 1 mM EDTA), 25 μM Amplex UltraRed (AUR), 0.125 μM horseradish perioxidase, 125 μM dithiothreitol, and 1.25% (weight) tween; or includes 6.6 M guanidine hydrochloride solution, 10 mM dithio-bis-nitrobenzoic acid, and 1 mM dithiothreitol.
